(19) United States
(12) Patent Application Publication
(10) Pub. No.: US 2011/0021999 A1
(43) Pub. Date: Jan. 27, 2011

(54) FILTER FOR FLOW IMPROVEMENTS FOR DELIVERY OF BIOMATERIALS

(76) Inventors: Stanley Kowalski, III, Wilbraham, MA (US); Scott Keeley, Charlestown, RI (US)

Correspondence Address: O'Shea Getz P.C.;Suite 912, 1500 Main Street, Springfield, MA, 01115, US

(21) Appl. No.: 12/749,079

(22) Filed: Mar. 29, 2010

Related U.S. Application Data
(63) Continuation of application No. PCT/US08/78098, filed on Sep. 29, 2008.
(60) Provisional application No. 60/975,841, filed on Sep. 28, 2007.

(51) Int. Cl. A61M 5/31

(52) U.S. Cl. 604/190

(57) ABSTRACT

A flow delivery system includes a syringe and a needle and/or a catheter that delivers an aqueous solution of a material, e.g., a biomaterial alone or mixed with a fluid lubricant, into a body. A filter located within the body of the syringe includes a plurality of openings each of a predetermined size. As the solution travels through the syringe, the solution encounters the filter openings which break up or downsize any biomaterial particles larger than the size of the openings. Also, the filter openings allow any particles of the biomaterial originally smaller than the size of the openings to pass without any downsizing. The size of the filter openings is selected as a function of the size of the opening in the needle and/or catheter. The downsized particles then pass with any other non-downsized particles in a relatively unobstructed manner into the body through the needle and/or catheter.

### PRIORITY INFORMATION

This patent application is a continuation of PCT Patent Application No. PCT/US08/78098 filed Sep. 29, 2008, which claims priority from U.S. patent application Ser. No. 60/975,841 filed Sep. 28, 2007, both of which are hereby incorporated by reference.

### BACKGROUND OF THE INVENTION

This invention relates in general to a flow delivery system that delivers an aqueous solution containing a biomaterial or a mixture of a biomaterial and a biocompatible fluid lubricant into a body, and in particular to such a system that includes a filter that breaks up or downsizes particles of the biomaterial that are larger than desired (e.g., a relatively large agglomerated mass of the particles) for more effective delivery of the aqueous solution into the body.

Medical procedures often involve the non-surgical implanting of biomaterials into the body. An example is the injecting of a dermal filler material such as collagen through the use of a syringe and needle system. The biomaterial can be solid and load-bearing and is typically suspended as an aqueous solution of the biomaterial particles. The solution is then injected with a syringe through a needle. For precise placement of materials into the facial dermis, a very fine needle, e.g., 27 gauge (0.0075″ inside diameter or ID) to 30 gauge (0.0055″ ID), is preferred. These relatively small ID needles limit the size of the suspended particles that may pass through the needle orifice. The size of the particle will typically range between 1-20 microns (0.001 mm-0.02 mm) in length and less than 20 microns (0.02 mm) in width. It has been determined that larger particles are desirable in some situations, such as the containment of time release medication. The larger particles pose a problem when used with the smaller needles required in the facial derma. The larger particles can bridge or agglomerate resulting in clogging of the small orifice needle. Larger particles also result in a greater amount of force needed to translate the syringe plunger. The higher force may cause the surgeon to tremble and slight perturbations of the hand could result in scaring of the patient. Therefore, it is desirable to have applied forces equivalent to a low viscidity Newtonian fluid.

Other applications for implanting a biomaterial into the human body include use of the biomaterial as a bulking or augmenting agent in internal body tissue, such as the tissue that defines various sphincters, for example, in the urinary tract (specifically, in the urinary outflow of the bladder into the urethra) or in the lower esophageal area connecting the esophagus to the stomach. The malfunctioning of these sphincters is usually in the form of improper or incomplete closure of the sphincters, which leads to medical conditions such as urinary incontinence and gastroesophageal reflux disease (GERD) or heartburn, respectively. Treatment of these medical conditions may include injections of a viscous material dispersed in a solution, such as collagen, in the vicinity of the associated sphincter to augment or bulk up and fortify the tissue and thereby assist in the adequate closure of the corresponding sphincter for re-establishment of normal sphincter control. Still other applications for implanting a biomaterial such as collagen into the human body include various other body passages and tissues; for example, for correcting wrinkles not only in the facial denim but in other areas of the body as well.

In these applications it is known to inject the biomaterial, typically suspended in an aqueous solution, into the human body through use of a syringe together with an elongate needle and/or catheter. This type of flow delivery system may be used as a standalone device or in combination with an appropriate medical instrument, such as a cystoscope, endoscope or gastroscope, which are utilized to view the tissue in the affected area. However, as the length of the elongate needle and/or catheter increases, the amount of force required to properly deliver the suspended mass aqueous solution of biomaterial to the desired body tissue area also increases. With known flow delivery systems, this increased amount of required force can cause problems both with the extrusion of the biomaterial through the flow delivery system and also with the intrusion of the biomaterial into the tissue. Oftentimes poor intrusion into the body tissue is the result of poor extrusion through the flow delivery system.

There has been substantial research and experimentation in various chemical compositions to reduce plunger force in a syringe and needle and/or catheter flow delivery system. An area commonly researched is the ability to introduce lubricity between the particles through use of an aqueous suspension of a particulate biocompatible material and a biocompatible fluid lubricant. The biomaterial and lubricant are typically combined in a manner that results in a homogenous mixture. It is believed that the lubricant enhances flow in part by preventing particle to particle contact. See, e.g., U.S. Pat. No. 4,803,075. However, a disadvantage of the addition of the lubricant is that it reduces the content of the active component in solution.

What is needed is an improved flow delivery system for implanting a biomaterial into the human body, where the system includes a filter that breaks up or downsizes particles of the biomaterial that are larger than desired, to achieve a more effective delivery of the aqueous solution into the body.

### SUMMARY OF THE INVENTION

Briefly, according to an aspect of the present invention, a flow delivery system includes a syringe and a needle and/or a catheter that delivers an aqueous solution of a material, such as a biomaterial or a mixture of a biomaterial and a biocompatible fluid lubricant, into a body, preferably at a constant applied force. Preferably, a filter is located within the body of the syringe. The filter includes a plurality of openings, each of a predetermined size. As the aqueous solution containing the suspended biomaterial particles travels through the body of the syringe under an applied force, the solution encounters the openings in the filter which break up or downsize any particles of the biomaterial within the solution that are larger than the size of the openings. At the same time, the openings in the filter allow any particles of the biomaterial that are smaller than the size of the openings to pass without any downsizing. The size of the openings in the filter may vary and preferably is selected in dependence on the size of the opening or orifice in the needle and/or catheter. The downsized particles then pass together with any other non-downsized particles in a relatively unobstructed manner through the needle and/or catheter and its orifice and into the body.

The present invention has utility in that the filter breaks up any agglomerated biomaterial particle matter or mass into smaller particles of a specific size (i.e., that of the openings in the filter). This reduces the resistance to the flow of the aqueous solution through a flow delivery system that includes the filter, which also reduces the amount of force necessary to transport and expel the aqueous solution through the system and into a body.

These and other objects, features and advantages of the present invention will become more apparent in light of the following detailed description of preferred embodiments thereof, as illustrated in the accompanying drawings.

### BRIEF DESCRIPTION OF THE DRAWINGS

FIGS. 1A-1D illustrate various views of an embodiment of a known flow delivery system including a syringe and a needle and/or a catheter;

FIGS. 2A-2B illustrate various views of a syringe and needle/catheter flow delivery system that includes a filter located inside the body of the syringe according to the present invention;

FIG. 3 is a perspective view of one embodiment of the filter of FIGS. 2A and 2B;

FIG. 4 is a perspective view of a bundle of glass tubes prior to slicing the bundle to form a second embodiment of the filter of FIGS. 2A and 2B; and

FIG. 5 is a perspective view of the second embodiment of the filter of FIGS. 2A and 2B.

### DETAILED DESCRIPTION OF THE INVENTION

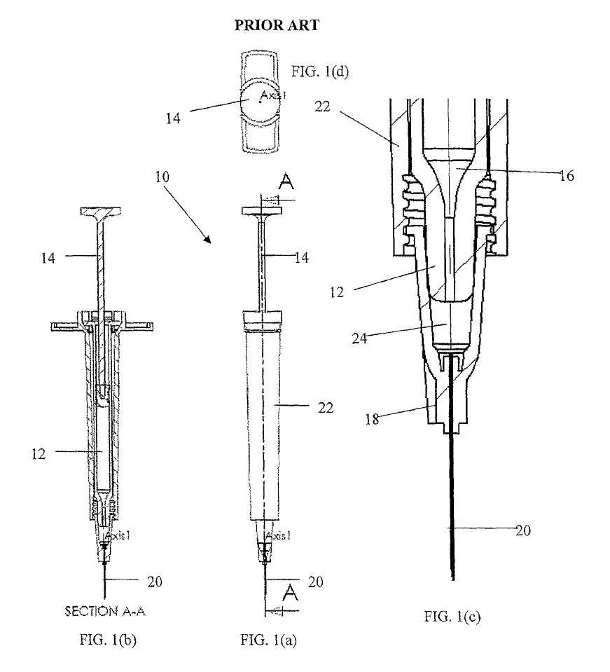

In the figures, like reference numerals refer to like elements. FIG. 1, including FIGS. 1A-1D illustrate various views of a known syringe and needle flow delivery system 10 for delivery of a material into a body. The material may comprise an aqueous solution of a biomaterial (e.g., a filler material such as collagen) alone or in a mixture with a biocompatible fluid lubricant. The biomaterial may comprise particles suspended in the solution. The syringe 12 is cylindrical in shape and may be made of glass or other suitable material and typically comprises a larger ID hollow inside section into which the plunger 14 fits. At the nozzle or discharge end of the syringe 12 is a tapered section 16 that significantly reduces the cross section of the syringe 12 at that location. A plastic housing 18 contains a needle 20, which may also include a catheter as an integral part thereof or as a separate component connected therewith, and is fitted over the syringe 12. The syringe/plastic housing assembly is held in place by an outer plastic sleeve 22 into which the plastic housing 18 is screwed. The syringe 12 is also held in place inside the outer plastic sleeve 22. A large plenum 24 is located between the exit opening of the syringe 12 and the distal opening of the needle 20. When the plunger 14 is pushed to create a force that injects the aqueous solution containing the biomaterial out of the needle 20 and into a body, the solution flows within the syringe 12 and is squeezed through the relatively narrow constriction at the exit of the syringe 12 past the tapered end 16 of the syringe 12. The solution will then expand into the larger plenum 24, and the solution is squeezed again to enter the needle 20 and then out into the body.

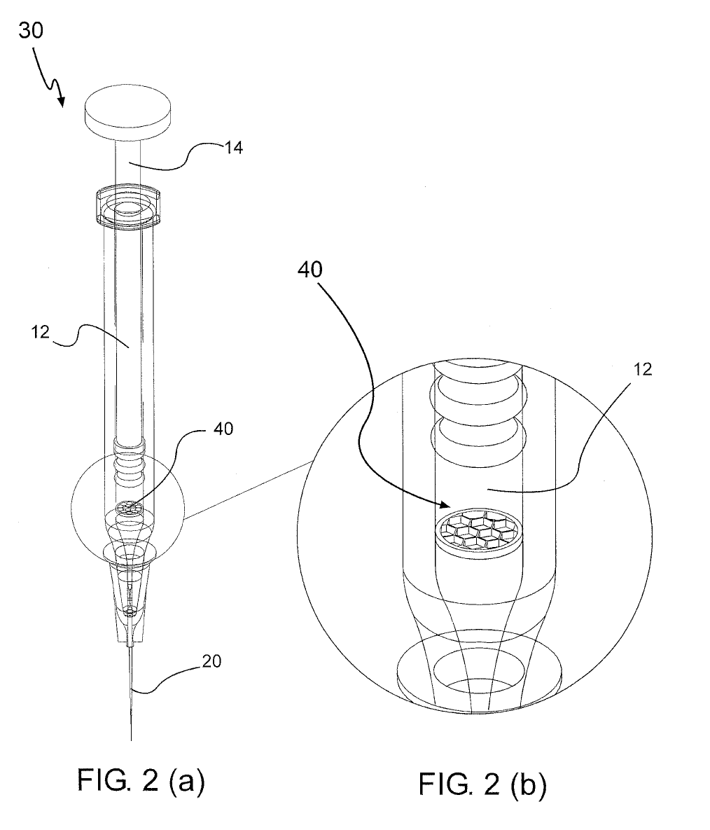

FIGS. 2A and 2B illustrate an embodiment of a flow delivery system 30 according to the present invention which reduces the amount of force required to transport and expel an aqueous solution of a biomaterial or a mixture of a biomaterial and a biocompatible fluid lubricant into a body at a desired location, such as, for example, the facial derma or a sphincter; specifically, the sphincter associated with the urinary tract or with the esophageal tract. The biomaterial may comprise collagen or other known materials used as bulking agents to augment or build up the tissue in the desired area to correct for improper sphincter operation or to cure cosmetic defects (e.g., wrinkles). The biocompatible fluid lubricant may comprise a non cross-linked collagen or other known materials that form a homogeneous mixture with the preferred biomaterial. Typically the amount of lubricant required in the mixture with the biomaterial is that which provides for proper intrudability of the biomaterial into the internal body tissue at the desired location and which also provides for proper extrudability of the biomaterial through and out from the flow delivery system 30.

The flow delivery system 30 may include the syringe 12, plunger 14 and needle and/or catheter 20, along with some or all of the other structural components of the known flow delivery system 10 of FIGS. 1A-1D, described in detail above. In the present embodiment, the flow delivery system 30 of the present invention includes a filter 40 located in the flow path inside the syringe 12 such that the filter 40 covers the entire cross-sectional area of the flow path inside the syringe 12. Also, the filter is illustrated as being located in the lower portion of the syringe 12 near the tapered end 16 of the syringe 12. However, the filter 40 may be located anywhere within the flow path inside of the syringe 12. The filter 40 may be adhered or press fit to the inner surface of the syringe 12. It suffices that the filter 40 be placed within the inside of the syringe 12 such that it does not move when the aqueous solution is forced through the syringe 12 by, e.g., the plunger 14.

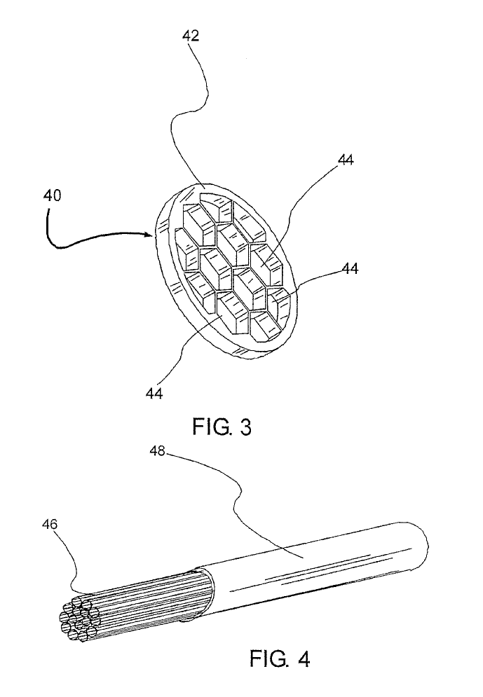

FIG. 3 illustrates a perspective view of an example of the filter 40 in FIGS. 2A and 2B. The filter 40 comprises a disk 42 having a plurality of through holes 44 of a predetermined shape formed in the disk 42 by, e.g., an etching process. In the exemplary embodiment of FIG. 3, the disk 42 may comprise a sterile material such as stainless steel, glass or other suitable material, and the plurality of through holes 44 all have a honeycomb shape and are of equal size. In the alternative, the size of the holes 44 may vary between one another. In one example, the size of the holes 44 are selected to depend on the size of the opening or orifice in the needle and/or catheter 20 utilized in the flow delivery system 30. An applied force, for example from the action of the plunger 14, propels the biomaterial particles within the aqueous solution through the holes 44 of the filter 40, thereby breaking up or downsizing the biomaterial particles larger than the holes 44. The downsized particles then pass through the holes 44 and through the reminder of the flow delivery system 30 and out of the needle/catheter 20 unobstructed and into a body. Also, particles smaller than the size of the holes 44 pass through the filter 40 without any downsizing.

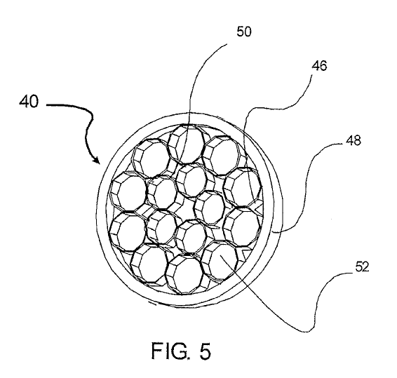

FIGS. 4-5 illustrate another example of the filter 40. In FIG. 4, a plurality of sterile solid glass tubes 46 are held bundled together by an outer sheath 48. The sheathed bundle of tubes 46 may then be sliced perpendicular to the axis of the tubes 46 to form the filter 40 of FIG. 5. The spaces 50 between the glass tubes and openings 52 within each tube function as the holes of the filter 40 for downsizing the particles within the aqueous solution. In another example, glass rods replace the glass tubes 46 to form a filter having spaces between the glass tubes. In this example, the openings 52 within the glass tubes 46 are of a predetermined size for downsizing of the particles within the aqueous solution and work in conjunction with the spaces 50 between the tubes 46 for downsizing of the particles.

In operation, the filter 40 within the flow delivery system 30 breaks up any agglomerated biomaterial particle matter or mass within the aqueous solution into smaller particles of a specific size or smaller (i.e., that of the openings 44 in the filter 40 of FIG. 3). Since the size of the openings in the filter 40 is selected in dependence on the size of the orifice of the needle/catheter 20 utilized, the size of the particles suspended in the aqueous solution and transported through the syringe 12 do not clog up the needle/catheter orifice. Instead, the solution is expelled without obstruction out of the orifice of the needle/catheter 20. Thus, the flow delivery system 30 of the present invention sizes the particulate matter within the syringe 12 before it reaches the needle 20. This reduces the resistance to the flow of the aqueous solution through the flow delivery system 30, which also reduces the amount of force necessary to transport and expel the aqueous solution through the system 30 and into a body, even with a system 30 that utilize an elongate needle/catheter 20.

The flow delivery system 30 of the present invention has been described for use with a conventional syringe and needle/catheter configuration that also contains a plunger 14 to supply a force to push the aqueous solution through the syringe 12 and out of the needle/catheter 20. However, the broadest scope of the present invention is not limited as such. The plunger 14 may be omitted and other means for forcing the aqueous solution through the syringe 12 may be utilized such as, for example, an acoustic transducer. Also, the syringe 12 may omit the plenum 24 (FIG. 1) and instead my employ various means for facilitating the flow of the aqueous solution out of the syringe and into and through the needles/catheter 20. For example, a contoured lower portion of the syringe 12 may be utilized.

Although the present invention has been illustrated and described with respect to several preferred embodiments thereof, various changes, omissions and additions to the form and detail thereof, may be made therein, without departing from the spirit and scope of the invention.

What is claimed is:

1. A system for delivery of a solution, comprising:
a syringe having a hollow inside portion in which the solution may be located and passes through under an applied force; and
a filter located within the hollow inside portion of the syringe, where the filter includes a plurality of holes each having a predetermined size, where the holes reduce in size any particles within the solution that are larger than the size of one or more of the plurality of holes that the larger size particles encounter as the solution passes through the syringe.

2. The system of claim 1, further comprising a plunger that provides the applied force to propel the solution through the syringe.

3. The system of claim 1, further comprising a needle connected to one end of the syringe through which the solution is expelled.

4. The system of claim 3, where the needle includes an orifice of a predetermined size, where the size of the plurality of holes in the filter depends on the size of the orifice in the needle.

5. The system of claim 1, further comprising a catheter connected to one end of the syringe through which the solution is expelled.

6. The system of claim 5, where the catheter includes an orifice of a predetermined size, where the size of the plurality of holes in the filter depends on the size of the orifice in the catheter.

7. The system of claim 1, further comprising a needle and catheter connected together and to one end of the syringe through which the solution is expelled.

8. The system of claim 1, where the filter comprises a disk of a size that equals an inside diameter of the hollow inside portion of the syringe.

9. The system of claim 1, where the plurality of holes in the filter are of the same size.

10. The system of claim 1, where the plurality of holes in the filter are of different sizes.

11. The system of claim 1, where each of the plurality of holes in the filter is of a honeycomb shape.

12. The system of claim 1, where the filter comprises a plurality of rods bundled together.

13. The system of claim 12, where an outer sheath surrounds the plurality of rods.

14. The system of claim 13, where each of the plurality of rods is solid, and where each of the plurality of holes in the filter comprises a spacing between at least two of the rods or between one of the rods and the outer sheath.

15. The system of claim 13, where each of the plurality of rods is hollow thereby forming a spacing within the rod, and where the plurality of holes in the filter comprises the spacing within each of the hollow rods.

16. The system of claim 13, where each of the plurality of rods is hollow thereby forming a spacing within the rod, and where the plurality of holes in the filter comprises the spacing within each of the hollow rods and a spacing between at least two of the rods or between one of the rods and the outer sheath.

17. The system of claim 1, where the filter comprises stainless steel.

18. The system of claim 12, where the rods comprise glass.

19. The system of claim 1, where the filter is adhered to an inside surface of the syringe.

20. The system of claim 1, where the filter is press fit within the hollow inside portion of the syringe.
